(19) United States
(12) Patent Application Publication
(10) Pub. No.: US 2009/0134785 A1
(43) Pub. Date: May 28, 2009

(54) ORGANOMETALLIC COMPLEX, ORGANIC LIGHT-EMITTING ELEMENT USING SAME, AND DISPLAY DEVICE

(75) Inventors: Jun Kamatani, Tokyo (JP); Masashi Hashimoto, Tokyo (JP); Satoshi Igawa, Fujisawa-shi (JP); Shinjiro Okada, Kamakura-shi (JP)

Correspondence Address: CANON U.S.A. INC. INTELLECTUAL PROPERTY DIVISION, 15975 ALTON PARKWAY, IRVINE, CA, 92618-3731, US

(73) Assignee: CANON KABUSHIKI KAISHA, Tokyo (JP)

(21) Appl. No.: 12/277,666

(22) Filed: Nov. 25, 2008

(30) Foreign Application Priority Data
Nov. 28, 2007 (JP) 2007-307693

(51) Int. Cl. H01J 1/63; C07D 213/89; C07F 17/00

(52) U.S. Cl. 313/504; 546/4; 546/6; 556/136

(57) ABSTRACT

An organometallic complex is represented by Formula (1) below.

MLₘL′ₙ (1)

In Formula (1) M stands for Ir, Rh, Pt, or Pd, m is an integer of 1 to 3, and n is an integer of 0 to 2, where m+n=3. MLₘ denotes a partial structure represented by Formula (2) below. ML′ₙ denotes a partial structure represented by at least one of Formulas (3) to (5) below.

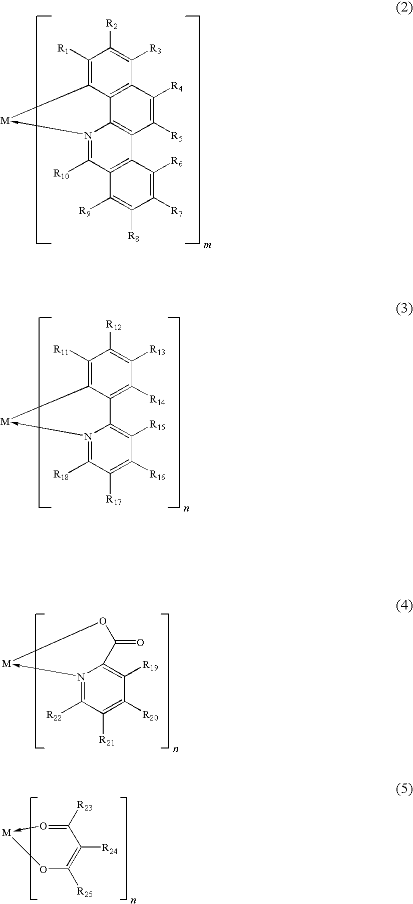

### BACKGROUND OF THE INVENTION

1. Field of the Invention

The present invention generally relates to an organometallic complex, an organic light-emitting element using the same, and a display device.

2. Description of the Related Art

An organic light-emitting element is generally an element in which a thin film containing a fluorescent organic compound is sandwiched between an anode and a cathode. Where electrons and holes (vacancies) are injected from respective electrodes, the fluorescent compound generates excitons, and the organic light-emitting element emits light when the excitons return to the ground state.

Significant progress has recently been achieved in the field of organic light-emitting elements, and they are now capable of featuring a high luminance at a low applied voltage, a large variety of emission wavelengths, and high-speed responsiveness, and have the potential for reducing the thickness and weight of light-emitting devices. Accordingly, a wide range of applications has been suggested for organic light-emitting elements.

However, there remains a need for a light output of even higher luminance and a higher conversion efficiency. Furthermore, there remains room for improvement with regards to durability, such as the change with time in long-term use and deterioration caused by oxygen-containing atmosphere gas or moisture.

Also, when applications to full-color displays are considered, good color purity and high-efficiency emission of red color may be required. Accordingly, a demand has been created for organic light-emitting elements of high color purity, emission efficiency, and durability and for materials for realizing such elements.

Iridium (Ir) complexes have been suggested as light-emitting materials that can use emission from a triplet state. Iridium complexes for use as light-emitting materials are disclosed in Macromol. Symp. 125, 1-48 (1997), “Improved energy transfer in electrophosphorescent device” (D. F. O'Brien et al., Applied Physics Letters Vol. 74, No. 3, p. 422 (1999)), “Very high-efficiency green organic light-emitting devices based on electrophosphorescence (M. A. Baldo et al., Applied Physics Letters, Vol. 75, No. 1, p. 4 (1999)), and Japanese Patent Laid-Open Nos. 2001-247859 and 2005-344124.

### SUMMARY OF THE INVENTION

In one embodiment, an organometallic complex in accordance with the present invention is represented by Formula (1) below.

MLₘL′ₙ (1)

In Formula (1), M stands for Ir, Rh, Pt, or Pd, m is an integer of 1 to 3, and n is integer of 0 to 2, where m+n=3. MLₘ denotes a partial structure represented by Formula (2) below. ML′ₙ denotes a partial structure represented by at least one of Formulas (3) to (5) below.

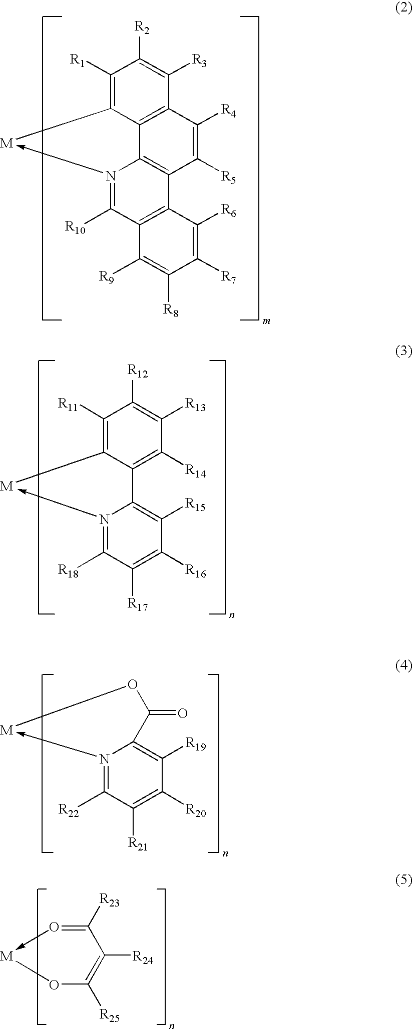

In Formulas (2) to (5), R₁ to R₂₅, which may be the same or different, are each a hydrogen atom, a halogen atom, a substituted or unsubstituted alkyl group, a substituted or unsubstituted alkoxy group, a substituted or unsubstituted aryloxy group, an aralkyl group, a substituted amino group, an aryl group, or a heterocyclic group. Any of the substituents R₁ to R₂₅ that are adjacent to one another may optionally be bonded to each other, thereby forming a ring.

Further features of the present invention will become apparent from the following description of exemplary embodiments with reference to the attached drawings.

### BRIEF DESCRIPTION OF THE DRAWINGS

FIG. 1 illustrates schematically a configuration example of a display device of one embodiment that includes an organic light-emitting element in accordance with the present invention and drive means.

FIG. 2 is circuit diagram illustrating an example of a circuit constituting one pixel disposed in the display device shown in FIG. 1.

FIG. 3 is a schematic diagram illustrating an example of a cross-sectional structure of a TFT substrate for use in the display device shown in FIG. 1.

FIG. 4 shows a PL spectrum of an Example Compound A-01 in a 1×10⁻⁵ mol/L toluene solution.

### DESCRIPTION OF THE EMBODIMENTS

An organometallic complex in accordance with one aspect of the present invention will be described below in greater details.

In one embodiment, the organometallic complex in accordance with the present invention is represented by Formula (1) below.

MLₘL′ₙ (1)

In Formula (1), M is Ir, Rh, Pt, or Pd.

In Formula (1), m is an integer of 1 to 3.

In Formula (1), n is an integer of 0 to 2, where m+n=3.

In Formula (1), L represents a bidentate. A specific structure thereof is described below.

In Formula (1), L′ represents a bidentate. However, L′ is not the same as L. A specific structure thereof is described below.

The bidentate ligand represented by L will be explained below. In one version, a specific partial structure represented by MLₘ is shown by Formula (2) below.

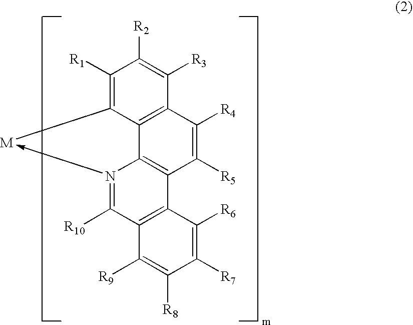

In Formula (2), R₁ to R₁₀ are each a hydrogen atom, a halogen atom, a substituted or unsubstituted alkyl group, a substituted or unsubstituted alkoxy group, a substituted or unsubstituted aryloxy group, an aralkyl group, a substituted amino group, an aryl group, or a heterocyclic group.

Examples of the halogen atom represented by R₁ to R₁₀ include fluorine, chlorine, bromine, and iodine.

Examples of the alkyl group represented by R₁ to R₁₀ include a methyl group, a trifluoromethyl group, an ethyl group, a normal propyl group, an isopropyl group, a normal butyl group, a tertiary butyl group, a secondary butyl group, an octyl group, a 1-adamantyl group, and a 2-adamantyl group. It goes without saying that this list is not limiting.

Examples of the alkoxy group represented by R₁ to R₁₀ include a methoxy group, an ethoxy group, a propoxy group, a 2-ethyl-octyloxy group, a trifluoromethoxy group, and a benzyloxy group. It goes without saying that this list is not limiting.

Examples of the aryloxy group represented by R₁ to R₁₀ include a phenoxy group, a 4-tert-butylphenoxy group, and a thienyloxy group. It goes without saying that this list is not limiting.

A benzyl group is an example of the aralkyl group represented by R₁ to R₁₀, but this example is not limiting.

Examples of the substituted amino group represented by R₁ to R₁₀ include an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, N,N-dimesitylamino group, N-phenyl-N-(4-tert-butylphenyl)amino group, and an N-phenyl-N-(4-trifluoromethylphenyl)amino group. It goes without saying that this list is not limiting.

Examples of the aryl group represented by R₁ to R₁₀ may include, but are not limited to, a phenyl group, a naphthyl group, an indenyl group, a pyrenyl group, an indacenyl group, an acenaphthenyl group, a phenantolyl group, a fluoranthenyl group, a triphenylenyl group, a chrysenyl group, a naphthacenyl group, a perylenyl group, a biphenyl group, a terphenyl group, and a fluorenyl group.

Examples of the heterocyclic group represented by R₁ to R₁₀ include a pyridyl group, an oxazolyl group, an oxadiazolyl group, a thiazolyl group, a thiadiazolyl group, a carbazolyl group, an acridinyl group, and a phenanthrolyl group. It goes without saying that this list is not limiting.

Examples of substituents that may be contained in the alkyl group, alkoxy group, and aryloxy group may include, but are not limited to, alkyl groups such as a methyl group, an ethyl group, and a propyl group; aralkyl groups such as a benzyl group; aryl groups such as a phenyl group and a biphenyl group; heterocyclic groups such as a pyridyl group and a pyrrolyl group; amino groups such as a dimethylamino group, a diethylamino group, a dibenzylamino group, a diphenylamino group, and a ditolylamino group; alkoxyl groups such as a methoxyl group, an ethoxyl group, and a propoxyl group; aryloxyl groups such as a phenoxyl group; halogen atoms such as fluorine, chlorine, bromine, and iodine; and a cyano group.

The substituents represented by R₁ to R₁₀ may be the same or different. Among the substituents represented by R₁ to R₁₀, in one version the adjacent substituents may also optionally be bonded to each other, thereby forming a ring such as a benzene ring, a cyclohexyl ring, and a pyridine ring.

The bidentate ligand represented by L′ will be explained below. In one version, a specific partial structure represented by ML′ₙ is shown by at least one of Formulas (3) to (5) below.

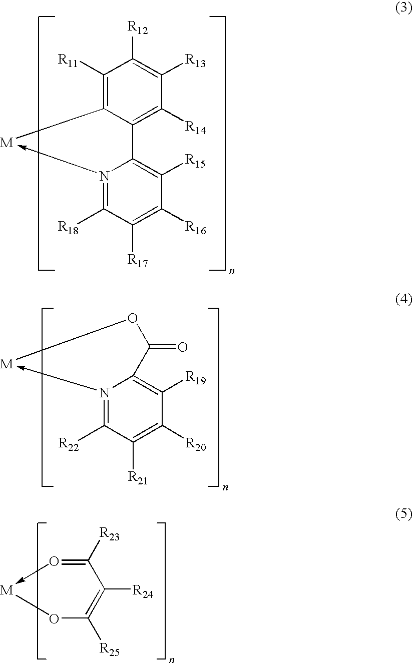

In Formulas (3) to (5), R₁₁ to R₂₅ are each a hydrogen atom, a halogen atom, a substituted or unsubstituted alkyl group, a substituted or unsubstituted alkoxy group, a substituted or unsubstituted aryloxy group, an aralkyl group, a substituted amino group, an aryl group, or a heterocyclic group.

Examples of the halogen atom represented by R₁₁ to R₂₅ include fluorine, chlorine, bromine, and iodine.

Examples of the alkyl group represented by R₁₁ to R₂₅ include a methyl group, an ethyl group, a normal propyl group, an isopropyl group, a normal butyl group, a tertiary butyl group, a secondary butyl group, an octyl group, a 1-adamantyl group, and a 2-adamantyl group. It goes without saying that this list is not limiting.

Examples of the alkoxy group represented by R₁₁ to R₂₅ include a methoxy group, an ethoxy group, a propoxy group, a 2-ethyl-octyloxy group, and a benzyloxy group. It goes without saying that this list is not limiting.

Examples of the aryloxy group represented by R₁l to R₂₅ include a phenoxy group, a 4-tert-butylphenoxy group, and a thienyloxy group. It goes without saying that this list is not limiting.

A benzyl group is an example of the aralkyl group represented by R₁₁ to R₂₅, but this example is not limiting.

Examples of the substituted amino group represented by R₁₁ to R₂₅ include an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group. It goes without saying that this list is not limiting.

Examples of the aryl group represented by R₁₁ to R₂₅ may include, but are not limited to, a phenyl group, a naphthyl group, an indenyl group, a pyrenyl group, an indacenyl group, an acenaphthenyl group, a phenantolyl group, a fluoranthenyl group, a triphenylenyl group, a chrysenyl group, a naphthacenyl group, a perylenyl group, a biphenyl group, a terphenyl group, and a fluorenyl group.

Examples of the heterocyclic group represented by R₁₁ to R₂₅ include a pyridyl group, an oxazolyl group, an oxadiazolyl group, a thiazolyl group, a thiadiazolyl group, a carbazolyl group, an acridinyl group, and a phenanthrolyl group. It goes without saying that this list is not limiting.

Examples of substituents that may be contained in the alkyl group, alkoxy group, and aryloxy group may include, but are not limited to, alkyl groups such as a methyl group, an ethyl group, and a propyl group; aralkyl groups such as a benzyl group; aryl groups such as a phenyl group and a biphenyl group; heterocyclic groups such as a pyridyl group and a pyrrolyl group; substituted amino groups such as a dimethylamino group, a diethylamino group, a dibenzylamino group, a diphenylamino group, and a ditolylamino group; alkoxyl groups such as a methoxyl group, an ethoxyl group, and a propoxyl group; aryloxyl groups such as a phenoxyl group; halogen atoms such as fluorine, chlorine, bromine, and iodine; and a cyano group.

The substituents represented by R₁₁ to R₂₅ may be the same or different.

Among the substituents represented by R₁₁ to R₂₅, in one version the adjacent substituents may also optionally be bonded to each other, thereby forming a ring such as a benzene ring, a cyclohexyl ring, and a pyridine ring.

In one embodiment, the organometallic complex in accordance with the present invention can be synthesized via a process of synthesizing the ligand represented by Formula (2) above, and a process of synthesizing a complex by reacting a metal atom and the ligand.

In one version, the ligand represented by Formula (2) above can be synthesized by referring to J. Org. Chem., 1988, 53, 1708-1713. More specifically, the synthesis can be conducted by the method represented by a Synthesis Route 1 shown below by using a benzaldehyde derivative with a halogen atom in a 2 position and a naphthylamine derivative as starting materials.

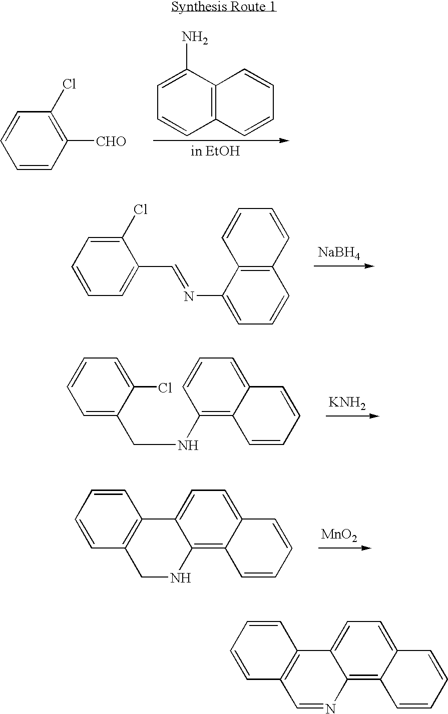

In the Synthesis Route 1, the benzaldehyde derivative serving as a starting material may have a substituent such as an alkyl group, a halogen atom, and a phenyl group in the benzene ring thereof. The naphthylamine derivative serving as a starting material may also have a substituent such as an alkyl group, a halogen atom, and a phenyl group in the naphthalene ring thereof.

Examples of ligands that can be synthesized using the Synthesis Route 1 are presented in the following table together with benzaldehyde derivatives and naphthylamine derivatives serving as starting materials.

TABLE 1

| | Benzaldehyde derivative | Naphthylamine derivative | Synthesized ligand |
|---|---|---|---|
| 1 | 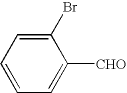 | 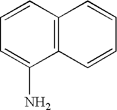 | 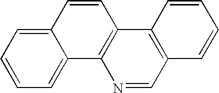 |
| 2 | 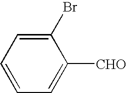 | 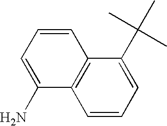 | 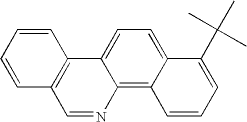 |
| 3 | 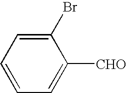 | 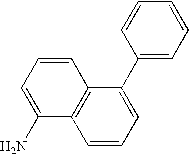 | 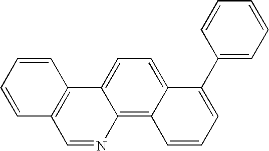 |
| 4 | 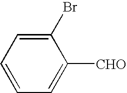 | 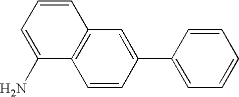 | 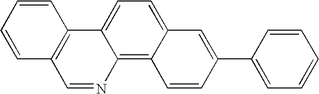 |
| 5 | 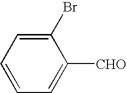 | 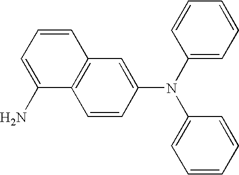 | 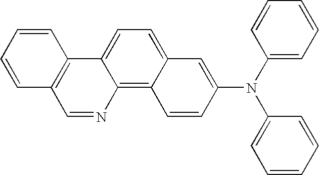 |
| 6 | 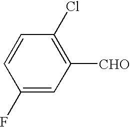 | 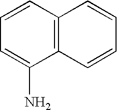 | 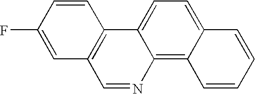 |
| 7 | 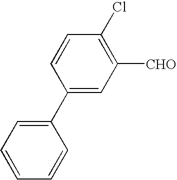 | 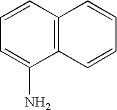 | 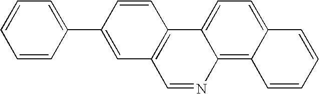 |
| 8 | 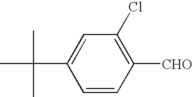 | 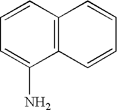 | 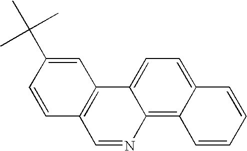 |

In one version, the organometallic complex in accordance with the present invention can be obtained by reacting the ligand thus obtained with a metal atom. More specifically, the organometallic complex in accordance with the present invention can be synthesized using the method represented by Synthesis Route 2 or Synthesis Route 3 below.

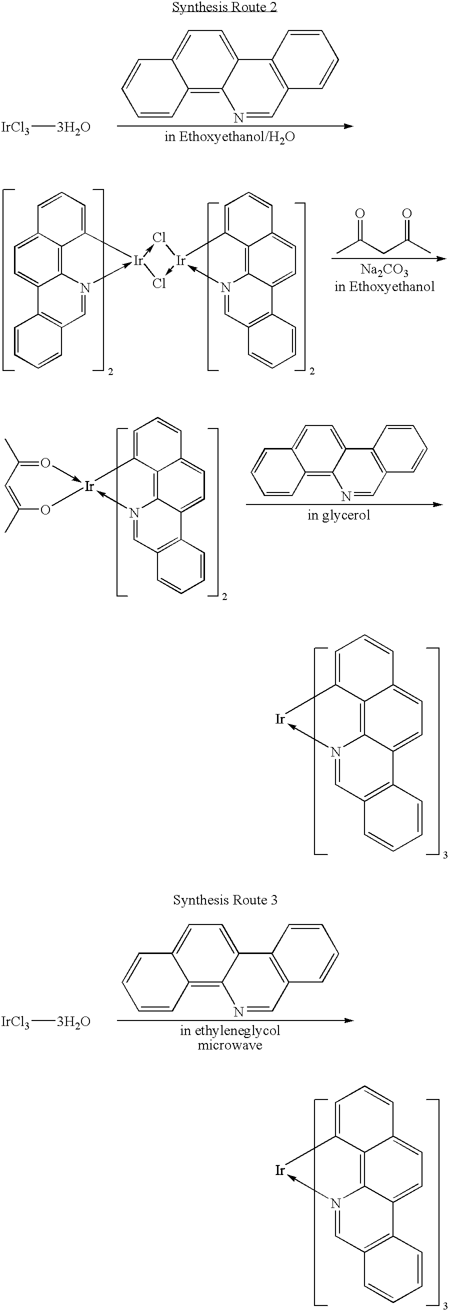

In one version, where the Synthesis Route 2 is used, an organometallic complex composed of ligands of two types can be synthesized using, for example, a picoline acid or tert-butyl acetylacetone instead of acetylacetone in the reaction of the second stage. Furthermore, in one version an organometallic complex composed of ligands of two types can be synthesized using, for example, a ligand such as phenyl pyridine instead of the ligand having a benzo[c]phenanthridine skeleton represented by Formula (2) in the reaction of the third stage.

In one embodiment, the organometallic complex in accordance with the present invention is a complex having a ligand with benzo[c]phenanthridine as a main skeleton. For example, the benzo[c]phenanthridine derivative may be a compound demonstrating light emitting ability. Furthermore, by contrast with phenyl isoquinoline represented by the formula below, in terms of the compound structure, benzo[c]phenanthridine does not have a single bond that can be a reason for a free rotation of sites relating to light emission.

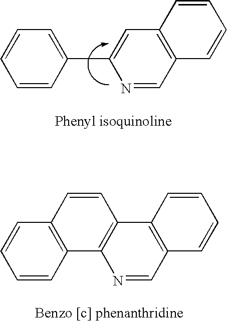

Therefore, according to one aspect of the invention, where a complex has a ligand with benzo[c]phenanthridine, and in particular when the complex has Ir as the central metal, deactivation during light emission can be reduced.

Without being limited to any particular theory herein, it is believed that nonradiative processes such as collisional deactivation with a transition to a ground state, vibrational relaxation with a transition to an adjacent vibrational level, and rotational relaxation with a transition to an adjacent rotational level are reasons for a decrease in light emission efficiency of light-emitting molecules. Accordingly, in view of these reasons for a decrease in light emission efficiency, the benzo[c]phenanthridine derivative, in which nothing can cause a free rotation of sites relating to light emission, is believed to be capable of reducing and even eliminating the decrease in light emission efficiency that may be associated with rotational deactivation (rotational relaxation). Therefore, where the organometallic complex in accordance with the present invention that has the benzo[c]phenanthridine derivative is used as a structural material of an organic light-emitting element, the light emission efficiency of the element may be increased.

On the other hand, when a light-emitting material is used as a constituent material for a display, light emission of red color (R), green color (G), and blue color (B) may be required. Similarly to the benzo[c]phenanthridine, benzoquinoline shown hereinbelow does not have structural reasons for a free rotation of sites relating to light emission. However, the benzoquinoline itself emits yellow light. Therefore, with respect to obtaining red light emission, benzoquinoline derivatives may not be considered as advantageous ligands.

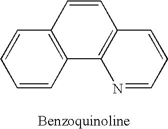

By contrast, benzo[c]phenanthridine has an emission color of 580 nm to 630 nm that enables the usage thereof as a red-light-emitting material. Accordingly, with regard to obtaining red light emission, benzo[c]phenanthridine derivatives may be exceptionally useful ligands.

Furthermore, in one version, an organometallic complex having a ligand with benzo[c]phenanthridine as the main skeleton, in particular when the central metal is Ir, may make it possible to obtain red light emission having a high emission efficiency due to a heavy atom effect.

In one embodiment, the organometallic complex in accordance with the present invention may have at least one ligand with benzo[c]phenanthridine as the main skeleton. However, it may also be the case that a plurality of such ligands are coordinated to the central metal.

In one version, a substituent for imparting a steric hindrance can be introduced in the ligand with benzo[c]phenanthridine as the main skeleton that constitutes the organometallic complex in accordance with the present invention. By introducing a substituent that imparts a steric hindrance, it may be possible to improve solubility of the ligand when the complex is synthesized. Furthermore, because the introduction of a substituent that imparts a steric hindrance inhibits concentration quenching, high-concentration doping may be possible when the complex is used as a constituent material for a light-emitting element, and an increase in light emission efficiency may be achieved.

Examples of substituents for imparting a steric hindrance may include, but are not limited to, substituents such as a methyl group, a tert-butyl group, and a phenyl group, that act to prevent the light-emitting ligands from coming excessively close to each other, and substituents such as halogen atoms that cause the repulsion of the molecules. By introducing such substituents, it may be possible to achieve light emission without excessive decrease in light emission efficiency, even in the case of high-concentration doping to a level of 5 wt. % or higher, based on the matrix.

Furthermore, while in one version the organometallic complex in accordance with the present invention may have a molecular structure in which ligands of the same type are coordinated to a metal atom, in another version it may also be the case that the organometallic complex has a molecular structure in which ligands of two types of different structure are coordinated, thereby making it possible to control the molecular weight or emission wavelength.

In one version, when the complex has a structure in which ligands of the same type are coordinated to a metal atom, a high symmetry with respect to the metal atom may ensure high thermal stability, thereby preventing the complex from decomposing easily during vapor deposition or the like, and providing for increased electric stability. On the other hand, in the version when the complex has a structure in which ligands of two kinds of different structure are coordinated with the metal atom, the molecular weight of the complex can be controlled, the vapor deposition temperature can thus be regulated by controlling the molecular weight, and the emission wavelength can also be controlled.

For example, when the central metal is Ir, the molecular weight of an organometallic complex in which three benzo[c]phenanthridine structures are coordinated as ligands is 877.02. By contrast, an organometallic complex in which two benzo[c]phenanthridine structures and one phenyl pyridine are coordinated has a molecular weight of 802.94. On the other hand, the molecular weight of an organometallic complex in which one benzo[c]phenanthridine and two phenyl pyridine structures are coordinated is 728.86. Because the molecular weight is thus decreased when the ligand is changed from benzo[c]phenanthridine to phenyl pyridine, the vapor deposition temperature may also be reduced.

Furthermore, when the complex has a structure in which two ligands of different structure are coordinated with a metal atom, the ligand making contribution to light emission can be appropriately adjusted. Therefore, inhibition of concentration quenching can be achieved, and the complex may be capable of emitting light, while inhibiting the decrease in light emission efficiency, even when the complex is doped to a concentration range of 10 wt. % or higher to 100 wt. %.

Further, the organometallic complex in accordance with the present invention can sterically be in the form of two structural isomers: a fac isomer and a mer isomer. The organometallic complex in accordance with the present invention may be of any of these structures, such as for example the fac isomer that typically ensures a high quantum yield. Where the complex has a structure in which ligands of two kinds of different structures are coordinated to a metal atom, a high quantum yield is sometimes obtained, for example, in the case of Ir(ppy)₂acac. Therefore, the fac isomer may not always be preferred. Furthermore, when the complex is synthesized, a specific structural isomer may be difficult to synthesize selectively and a mixture of two isomers can be used to reduce the cost.

Specific examples of the organometallic complex in accordance with the present invention are shown below. However, the present invention is not intended to be limited to these examples.

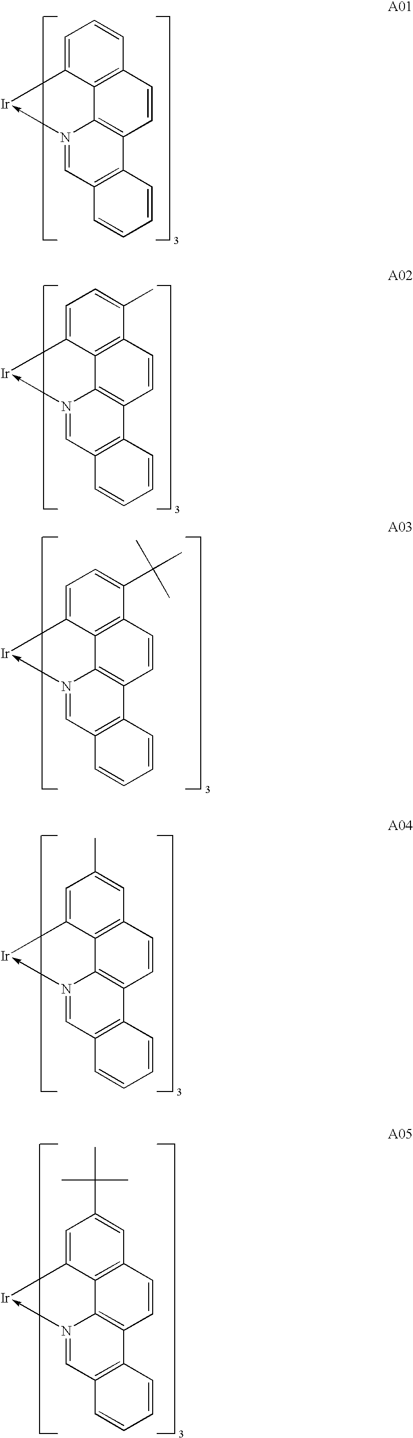

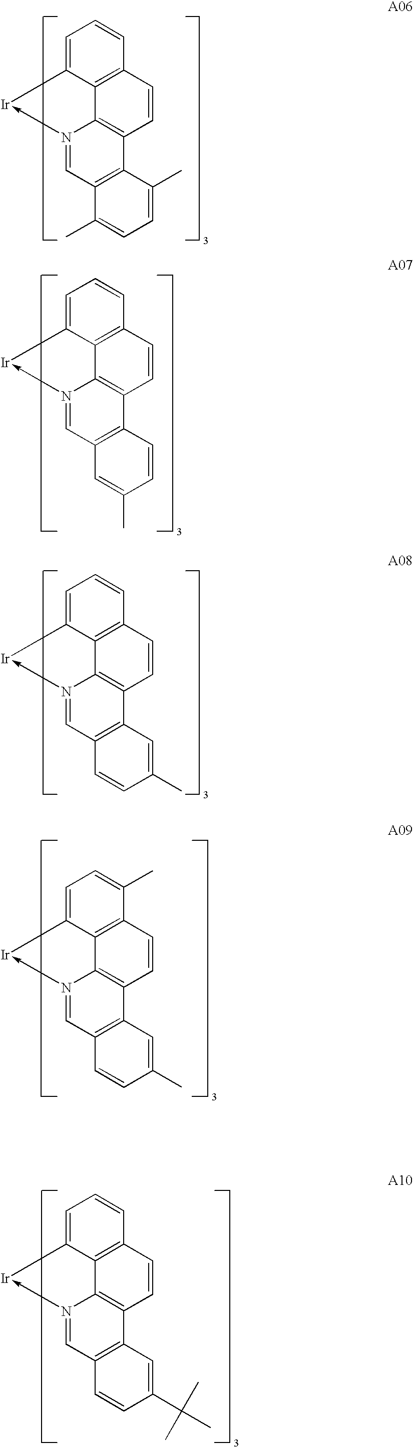

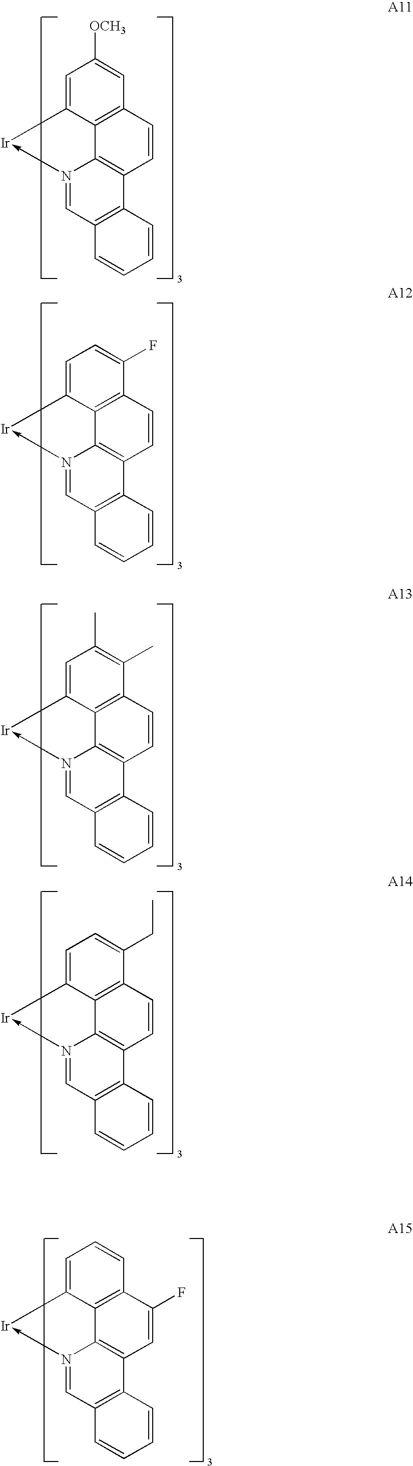

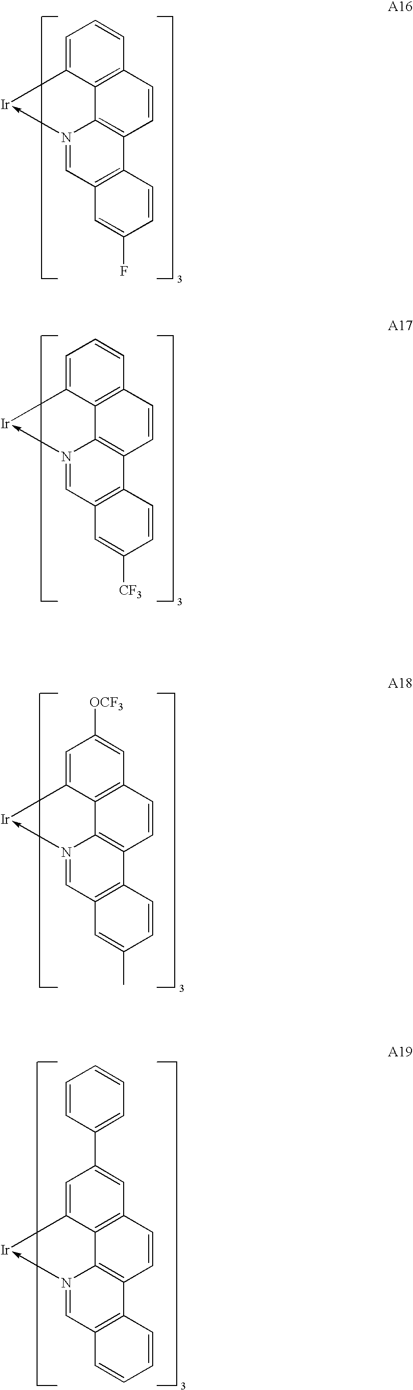

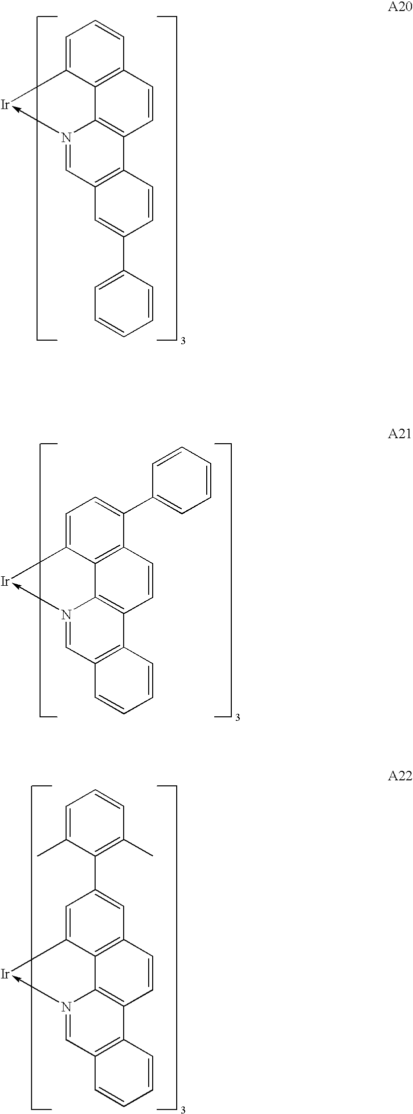

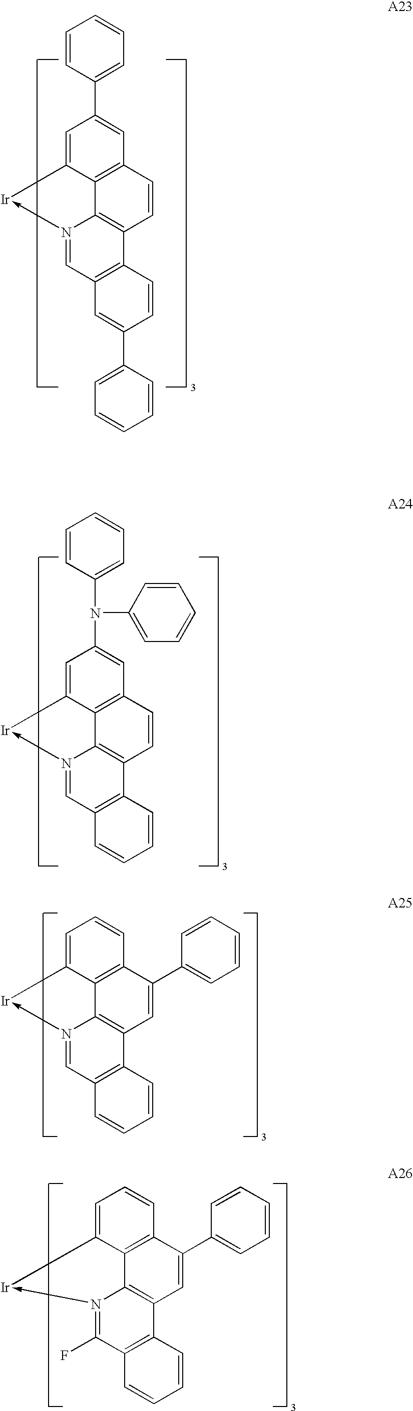

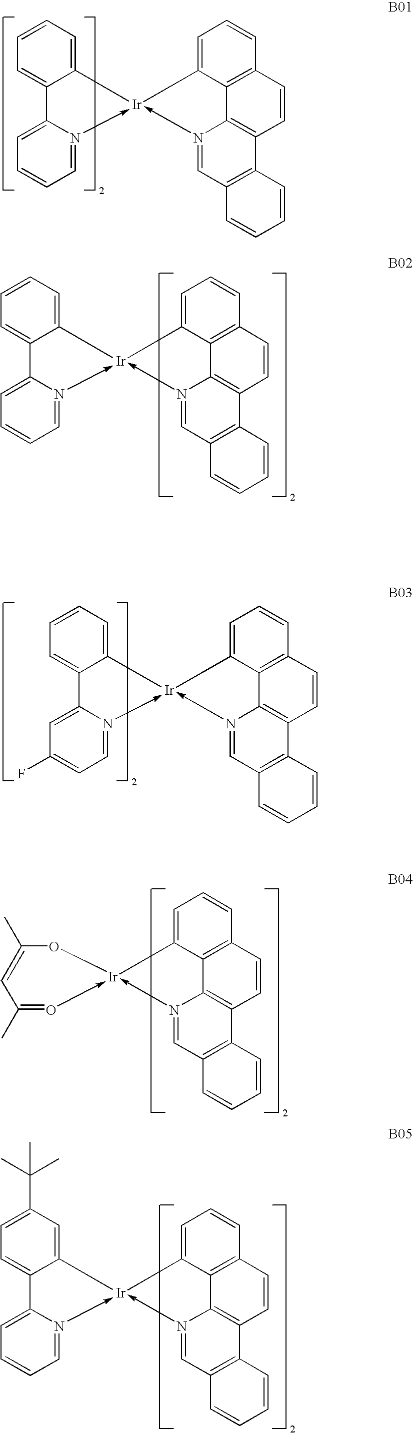

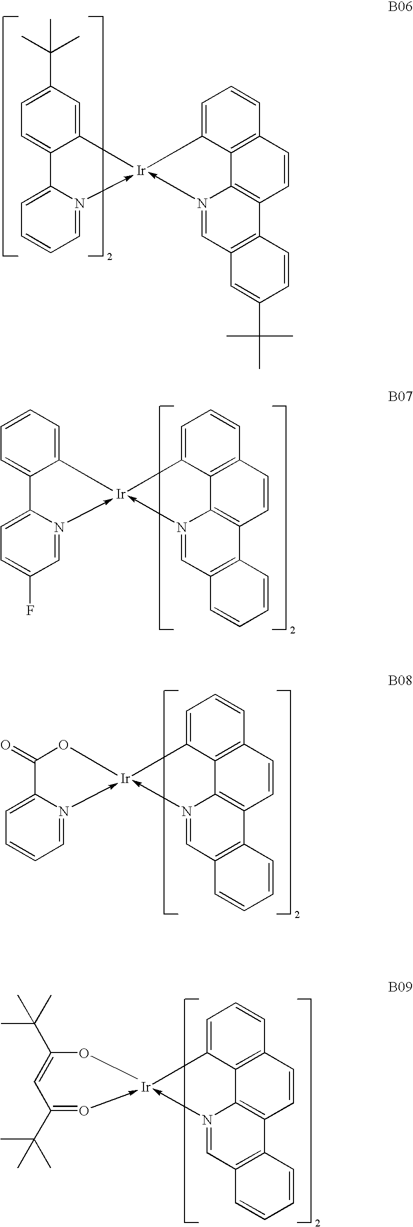

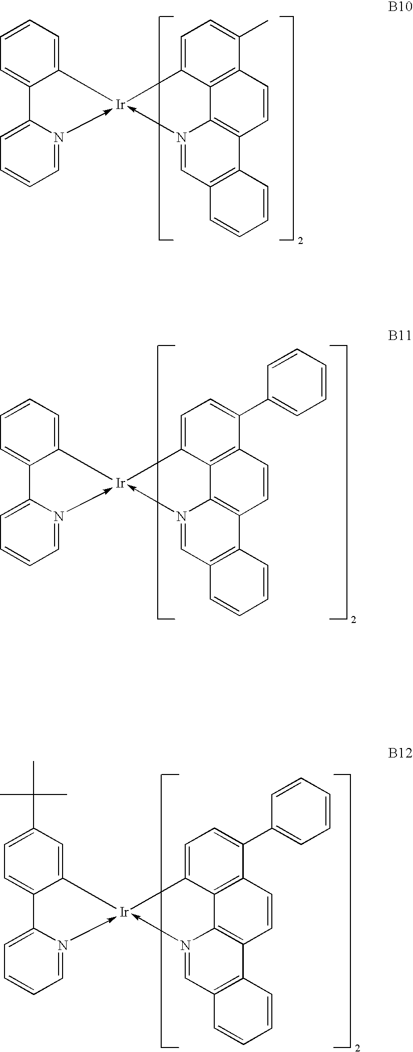

Aspects of a organic light-emitting element in accordance with the present invention will be described below.

In one version, the organic light-emitting element in accordance with the present invention comprises an anode, a cathode, and an organic compound sandwiched between the anode and the cathode.

Embodiments of the organic light-emitting element in accordance with the present invention will be described below.

In a first embodiment of the organic light-emitting element in accordance with the present invention, an anode, a light-emitting layer, and a cathode are provided in the order of description on a substrate. The first embodiment may be useful, for example, in the case where the light-emitting layer is configured by an organic compound that combines the hole transport ability, electron transport ability, and light emitting ability. Furthermore, this embodiment may also be useful in the case where the light-emitting layer is configured by mixing compounds each having a property selected from the hole transport ability, electron transport ability, and light emitting ability.

In a second embodiment of the organic light-emitting element in accordance with the present invention, an anode, a hole transport layer, an electron transport layer, and a cathode are provided in the order of description on a substrate. The organic light-emitting element of the second embodiment may be useful, for example, when a light-emitting organic compound that is a light-emitting substance having any of hole transport ability and electron transport ability is used in combination with an organic compound having only the electron transport ability or only the hole transport ability. Further, in the organic light-emitting element of the second embodiment, the hole transport layer or electron transport layer also serves as a light-emitting layer.

In a third embodiment of the organic light-emitting element in accordance with the present invention, an anode, a hole transport layer, a light-emitting layer, an electron transport layer, and a cathode are provided in the order of description on the substrate. In the organic light-emitting element of the third embodiment, the carrier transport function and the light emission function are separated, and organic compounds having respective characteristics from among the hole transport ability, electron transport ability, and light emission ability can be used in appropriate combinations. As a result, in the third embodiment, the degree of freedom in selecting suitable materials may be increased and various compounds with different emission wavelengths can be used. Therefore, the variety of emission hues may be increased. Further, it may also be possible to effectively confine the carriers or excitons to the central light-emitting layer, thereby increasing the emission efficiency of the organic light-emitting element.

In a fourth embodiment of the organic light-emitting element in accordance with the present invention, an anode, a hole injection layer, a hole transport layer, a light-emitting layer, an electron transport layer, and a cathode are provided in the order of description on a substrate. In the organic light-emitting element of the fourth embodiment, because the hole injection layer is provided between the anode and the hole transport layer, adhesion and hole injection ability can be improved. Therefore, the voltage supplied may be effectively reduced.

In a fifth embodiment of the organic light-emitting element in accordance with the present invention, an anode, a hole transport layer, a light-emitting layer, a hole/exciton blocking layer, an electron transport layer, and a cathode are provided in the order of description on a substrate. In the fifth embodiment, the hole/exciton blocking layer, which is a layer that blocks the penetration of holes or excitons to the cathode side, is provided between the light-emitting layer and the electron transport layer. By using a compound with a very high ionization potential as a constituent material for the hole/exciton blocking layer, it may be possible to increase the emission efficiency.

The above-described first to fifth embodiments represent very basic element configurations, and the configuration of the organic light-emitting element in accordance with the present invention is not limited to these configurations. For example, a large variety of layered structures can be employed that have, for example, an insulating layer, an adhesive layer, or an interference layer on the interfaces of electrodes and organic compound layer, and in which, for example, a hole transport layer is composed of two layers with different ionization potentials.

In the organic light-emitting element in accordance with the present invention, the organometallic complex in accordance with the present invention can be used in any of the first to fifth embodiments, as well as in other embodiments. In one version, in the organic light-emitting element in accordance with the present invention, the organometallic complex in accordance with the present invention is contained in a layer comprising an organic compound (organic compound layer). The organic compound layer as referred to herein may be, for example, any of the hole injection layer, hole transport layer, light-emitting layer, hole/exciton blocking layer, and electron transport layer described in the first to fifth embodiments above, such as for example the light-emitting layer.

In one version of the organic light-emitting element in accordance with the present invention, the light-emitting layer may comprise only the organometallic complex in accordance with the present invention, however the light-emitting layer may also comprise both a host and a guest.

When the light-emitting layer of the organic light-emitting element comprises both a host and a guest having carrier transport ability, the following several processes are believed to mainly contribute to light emission, and energy transfer and light emission in each of these processes may occur in competition with a variety of deactivation processes.

(1) Transport of electrons and holes within the light-emitting layer.

(2) Host exciton generation and guest exciton generation 1.

(3) Excitation energy transfer between host molecules.

(4) Excitation energy transfer from the host to the guest, and guest exciton generation 2.

(5) Light emission from guest molecules.

Typically, in order to increase the light emission efficiency of an organic light-emitting element, it may be desirable that the emission quantum yield of the light-emitting central material is high.

Accordingly, in one aspect, as embodiments of the organometallic complex in accordance with the present invention have a relatively high quantum yield of light emission in a dilute solution, a relatively high light emission efficiency may be achieved when the organometallic complex in accordance with the present invention is used as a constituent material of an organic light-emitting element.

When the organometallic complex in accordance with the present invention is used as a guest (dopant) in the organic light-emitting element in accordance with the present invention, for example, one or more of an iridium compound, compounds shown in Table 2 below, and derivatives thereof, can be used as the corresponding host, but these examples are obviously not limiting.

TABLE 2

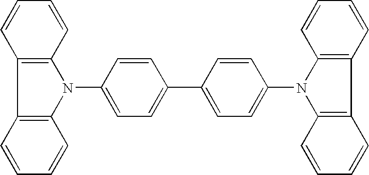

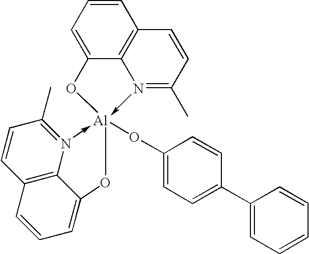

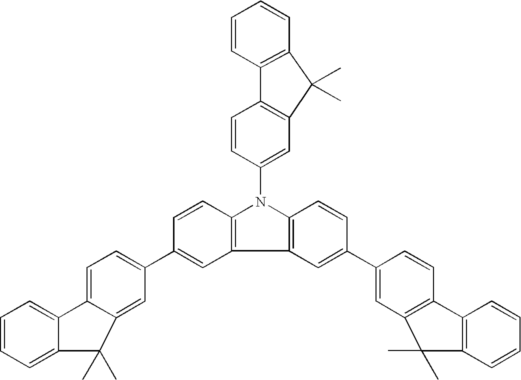

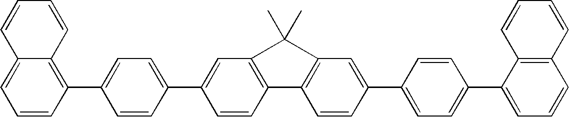

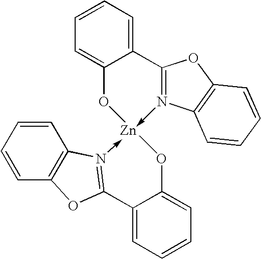

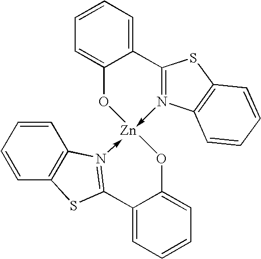

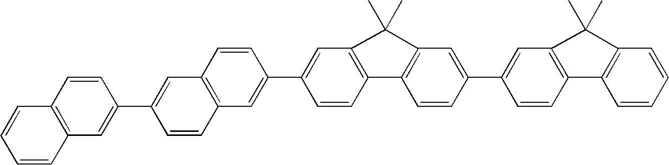

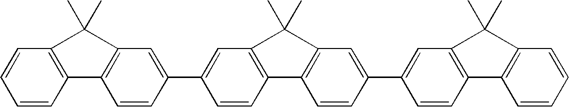

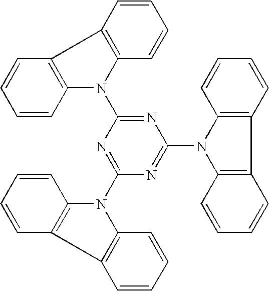

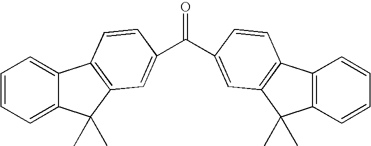

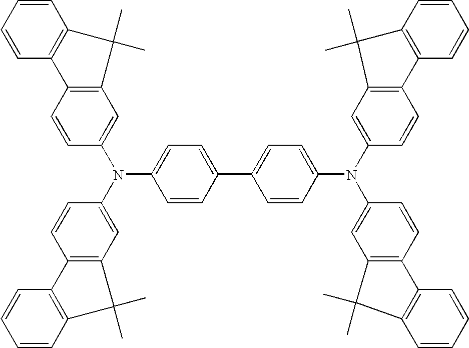

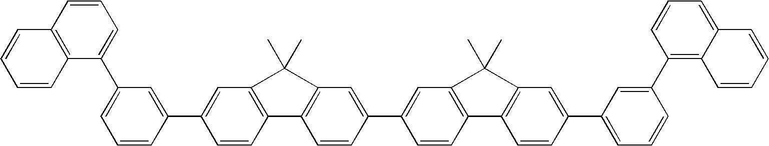

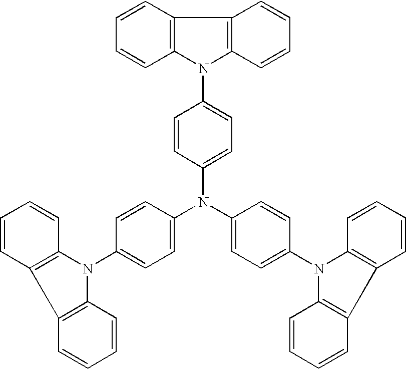

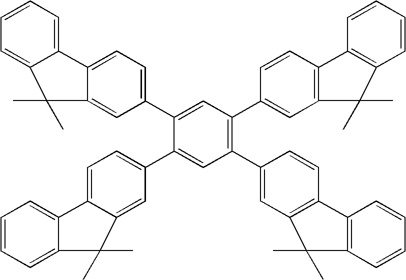

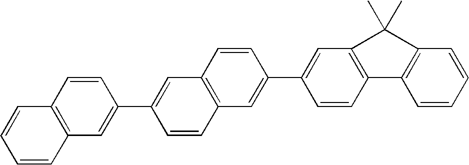

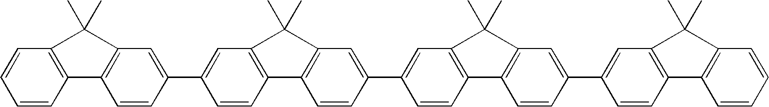

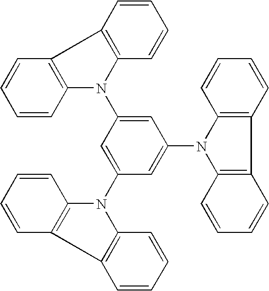

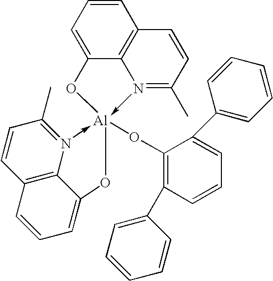

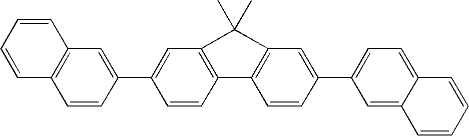

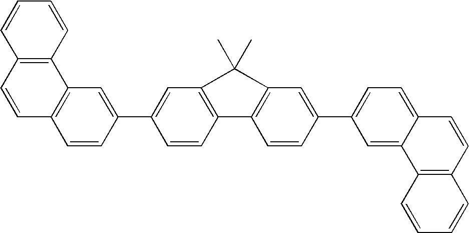

In one version, when the organometallic complex in accordance with the present invention is used as a guest (dopant), the concentration of the guest may be 0.01 wt. % to 20 wt. %, such as 0.5 wt. % to 10 wt. % based on the host. Further, by regulating the guest concentration it may be possible to increase the wavelength of light emission of the element to about 5 nm to 20 nm.

In another version, when the organometallic complex in accordance with the present invention is used as a host, the concentration of the corresponding guest may be 2 wt. % to 20 wt. % based on the host.

In one embodiment, the organic light-emitting element in accordance with the present invention specifically uses the organometallic complex in accordance with the present invention as a constituent material of the light-emitting layer. However, low-molecular and polymer hole injection/transport materials, light-emitting compounds, and electron injection/transport materials, such as those that have heretofore been well known, can also be used in combination therewith.

In one version, a material with a high hole mobility, such that it makes it possible to inject holes from the anode and transport the injected holes to the light-emitting layer, may be provided as the hole injection/transport material. Examples of low-molecular and polymer material having hole injection/transport capability include triarylamine derivatives, phenylenediamine derivatives, stilbene derivatives, phthalocyanine derivatives, porphyrin derivatives, poly(vinyl carbazole), poly(thiophene), and other electrically conductive polymers, but this list is by no way limiting.

In addition to the organometallic complex in accordance with the present invention, examples of the light-emitting compound can include Ir(ppy)₃, Pt(OEP), Ir(piq)₃, Alq₃, rubrene, and coumarine.

In another version, any compound that can easily inject electrons from the cathode, and transport the injected electrons to the light-emitting layer, can be selected as the electron injection/transport material, and the appropriate selection may be made with consideration for a balance with the hole mobility of the hole injection/transport material, and the like. Examples of materials having the electron injection/transport capability may include, but are not limited to, oxadiazole derivatives, oxazole derivatives, pyrazine derivatives, triazole derivatives, triazine derivatives, quinoline derivatives, quinoxaline derivatives, phenanthroline derivatives, and organoaluminum complexes.

In yet another version, a material with a high work function may be used as the material constituting the anode. Examples of suitable materials can include, but are not limited to, individual metals such as gold, platinum, silver, copper, nickel, palladium, cobalt, selenium, vanadium and tungsten, alloys thereof, or metal oxides such as tin oxide, zinc oxide, indium oxide, indium tin oxide (ITO), and indium zinc oxide. Further, conductive polymers such as polyaniline, polypyrrole, and polythiophene can be also used. These electrode substances may be used individually or in combination of two or more kinds thereof. The anode may also have a monolayer structure or a multilayer structure.

In a further version, a material with a low work function may be used as the material constituting the cathode. Examples of suitable materials can include, but are not limited to, individual metals such as alkali metals, e.g. lithium, alkaline earth metals, e.g. calcium, aluminum, titanium, manganese, silver, lead, and chromium. Alloys in which these metals are combined may be also used. For example, magnesium-silver, aluminum-lithium, and aluminum-magnesium alloys can be used. Metal oxides such as indium tin oxide (ITO) can be also used. These electrode substances may be used individually or in combination of two or more kinds thereof. The cathode may also have a monolayer structure or a multilayer structure.

The substrate used in accordance with the present invention is not particularly limited, and an opaque substrate such as for example a metallic substrate or a ceramic substrate, or a transparent substrate such as glass, quartz, or a plastic sheet can be used.

In one embodiment, color light emission can be controlled by using a color filter film, a fluorescent color conversion filter film, a dielectric reflective film, or the like on the substrate. Further, it may also be possible to produce a thin-film transistor (TFT) on a substrate and produce an element connected thereto. Yet another possible option may be to configure a matrix on a substrate, produce elements, and use them for illumination.

According to one aspect, the organic light-emitting element in accordance with the present invention can be applied to produce energy-saving device of high luminance. Examples of applications can include, but are not limited to image display devices, light sources for printers, illumination devices, and backlights of liquid crystal display devices.

Examples of image display devices can include, but are not limited to, energy-saving lightweight flat panel displays of high visibility.

As for light sources for printers, embodiments of organic light-emitting elements in accordance with the present invention can replace the laser light source units of laser beam printers that have been widely used in recent years. As a replacement method, for example, the organic light-emitting elements that can be addressed individually can be disposed on an array. Even when a laser light source unit is replaced with the organic light-emitting element in accordance with the present invention, it may be possible to form an image in the same manner as in the conventional configurations by exposing a photosensitive drum in a desired manner. By using the organic light-emitting element in accordance with the present invention, it may be possible to reduce significantly the volume of the device.

The utilization of the organic light-emitting element in illumination devices and backlights may also achieve an energy saving effect.

An embodiment of a display device using the organic light-emitting element in accordance with the present invention will be described below. This display device includes an organic light-emitting element in accordance with the present invention and means for supplying electric signals to the organic light-emitting element in accordance with the present invention. The embodiment of the display device in accordance with the present invention will be described below in greater details by considering an active matrix system by way of example with reference to the drawings. First, reference symbols shown in the figures will be explained: 1—a display device, 2 and 14—pixel circuits, 11—a scan signal driver, 12—an information signal driver, 13—a current supply source, 21—a first thin-film transistor (TFT 1), 22—a capacitor (C_{add}), 23—a second thin-film transistor (TFT 2), 31—a substrate, 32—a moisture-proof layer, 33—a gate electrode, 34—a gate insulating film, 35—a semiconductor film, 36—a drain electrode, 37—a source electrode, 38—a TFT element, 39—an insulating film, 310—a contact hole (through hole), 311—an anode, 312—an organic layer, 313—a cathode, 314—a first protective layer, and 315—a second protective layer.

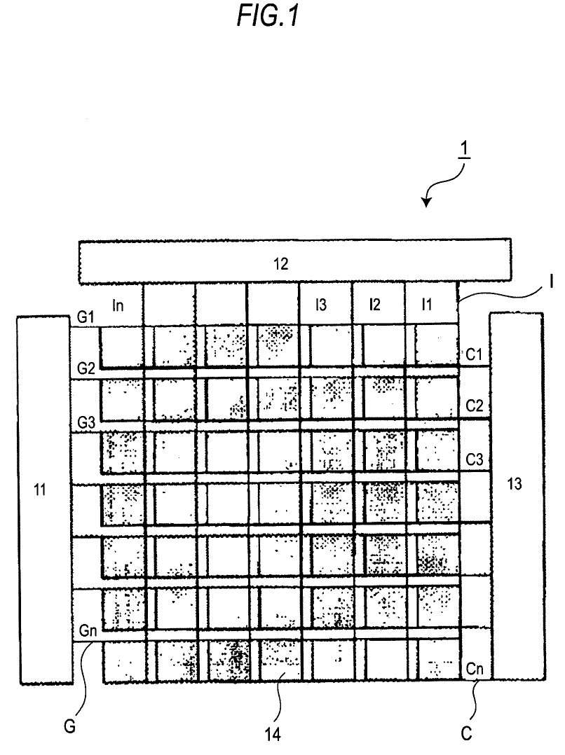

FIG. 1 illustrates schematically a configuration example of a display device of one embodiment including the organic light-emitting element in accordance with the present invention and drive means. The display device 1 shown in FIG. 1 has arranged therein the scan signal driver 11, information signal driver 12, and current supply source 13 that are connected to gate selection lines G, information signal lines I, and current supply lines C, respectively. A pixel circuit 14 is arranged in each intersection of the gate selection line G and information signal line I. The scan signal driver 11 successively selects gate selection lines G1, G2, G3, . . . Gn, and pixel signals from the information signal driver 12 are applied synchronously with this selection to the pixel circuits 14 via the information signal line I1, I2, I3, . . . In.

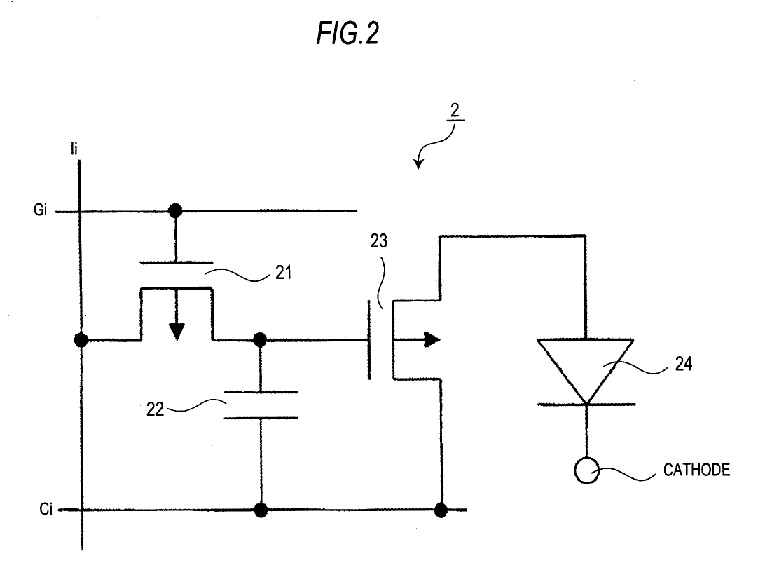

The operation of a pixel will be explained below. FIG. 2 is a circuit diagram illustrating an example of a circuit constituting one pixel disposed in the display device shown in FIG. 1. In the pixel circuit 2 shown in FIG. 2, where a selection signal is applied to the gate selection line Gi, the first thin-film transistor (TFT1) 21 is switched ON, an image signal Ii is supplied to the capacitor (C_{add}) 22, and the gate voltage of the second thin-film transistor (TFT 2) 23 is determined. An electric current is supplied to the organic light-emitting element 24 from the current supply line Ci in response to the gate voltage of the second thin-film transistor (TFT 2) 23. Here, the gate potential of the second thin-film transistor (TFT 2) 23 is held in the capacitor (C_{add}) until the first thin-film transistor (TFT 1) 21 performs the next scanning and selection. Therefore, the electric current continues to flow in the light-emitting element 24 until the next scan is performed. As a result, the light-emitting element 24 can be caused to emit light constantly within one frame period.

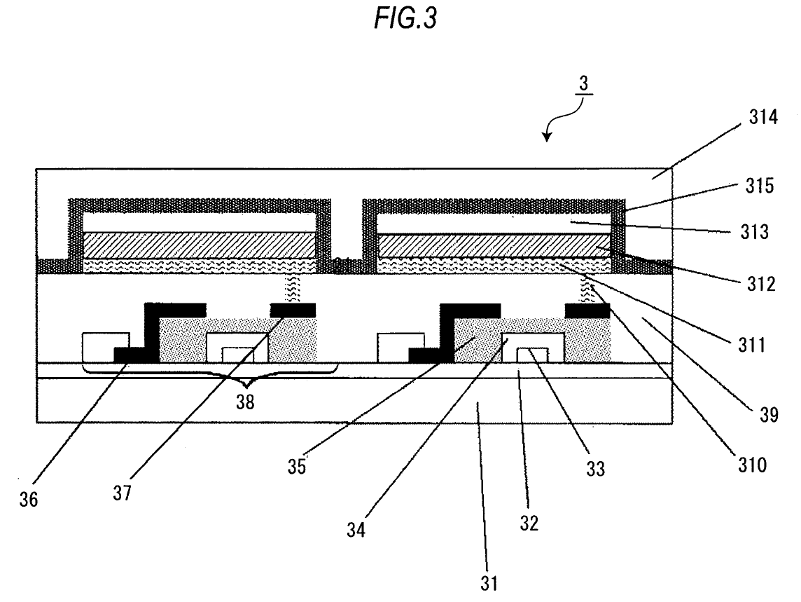

FIG. 3 is a schematic diagram illustrating an example of a cross-sectional structure of a TFT substrate for use in the display device shown in FIG. 1.

The structure will be explained below with reference to an example of a process for manufacturing the TFT substrate. According to this example, when the display device 3 shown in FIG. 3 is manufactured, first, a moisture-proofing film 32 is coated on a substrate 31, such as glass, to protect the components (TFT or organic layer) produced in the upper portion thereof. Examples of materials constituting the moisture-proofing film 32 may include, for example, silicon oxide and a composite of silicon oxide and silicon nitride. Then, a metal (e.g., Cr) film is produced by sputtering, thereby patterning a predetermined circuit shape and forming the gate electrode 33. Then, a film of silicon oxide or the like is produced by, for example, a plasma CVD method or a catalytic chemical vapor deposition method (cat-CVD method) and the film is patterned to form the gate insulating film 34. Then, a silicon film is produced by plasma CVD or the like (sometimes annealing is performed at a temperature equal to or higher than 290° C.), patterning is conducted according to the circuit shape, and the semiconductor layer 35 is formed.

The TFT element 38 of this example is then fabricated by providing the drain electrode 36 and the source electrode 37 on the semiconductor film 35, and a circuit such as shown in FIG. 2 is formed. The insulating film 39 is then formed on top of the TFT element 38. The contact hole (through hole) 310 is then formed so as to connect the metallic anode 311 and source electrode 37 for an organic light-emitting element.

In this example, the display device 3 can be obtained by successively laminating the multilayer or monolayer organic layer 312 and cathode 313 on the anode 311. In this case, the first protective layer 314 or second protective layer 315 may be provided to prevent the organic light-emitting element from deterioration. By driving the display device using the organic light-emitting element in accordance with the present invention, it may be possible to obtain stable display with good image quality even in long-term display.

Furthermore, the above-described display device in accordance with the invention is not limited to a switching element, and can be easily applied to, for example, a single-crystal silicon substrate, a MIM element, and an a-Si type.

### EXAMPLES

The present invention will be described below in greater details with reference to the Examples, but the present invention is not intended to be limited thereto.

### Example 1

### Synthesis of Example Compound A-01

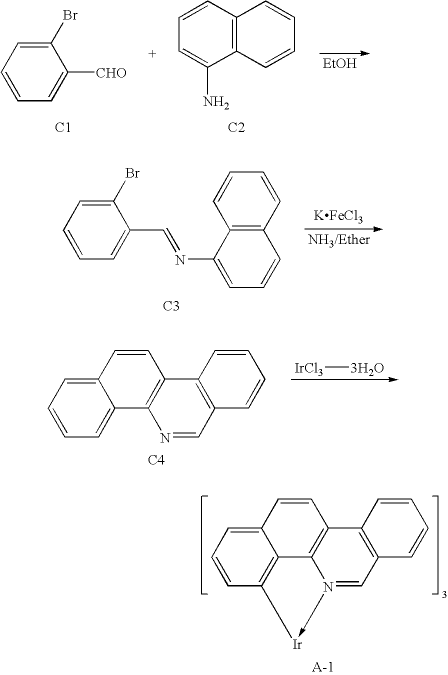

(1) The following reagents and solvent were charged into an eggplant type flask with a capacity of 300 mL.
Compound C1: 25 g (135 mmol).
Compound C2: 21.3 g (149 mmol).
Ethanol: 80 ml.

The reaction liquid was heated under stirring, and the heating was stopped when refluxing was started. The reaction solution was then cooled and the precipitated crystals were filtered at room temperature and dried to obtain 35.7 (yield 85%) of compound C3 in the form of yellow crystals.

(2) A total of 1200 ml of liquid ammonia was placed in a 2 L flask, and then 1.9 g of metallic potassium and 0.31 g of anhydrous iron chloride were charged, while maintaining the liquid temperature at −40° C. After the color of the reaction solution was confirmed to change to yellowish brown, 36.0 g (a total of 37.9 g, 970 mmol) of metallic potassium was additionally charged. Stirring was then performed for 1 h, while maintaining the temperature of the reaction liquid at −35° C, and then a diethyl ether solution obtained by dissolving the compound C3 (35.7 g) in 420 ml of dehydrated diethyl ether was dropwise added. Stirring was then performed for 1 h, while maintaining the temperature of the reaction liquid at −35° C. The temperature of the reaction solution was then raised to room temperature and ammonia present in the reaction solution was evaporated and removed. Saturated brine was then added to the reaction solution, and the organic layer was then cautiously extracted using THF. This organic layer was washed with saturated brine, dried with magnesium sulfate and then concentrated under reduced pressure, producing 23.4 g of a crude product in the form of yellowish brown crystals. The obtained crude product was purified by silica gel column chromatography (developing solvent: hexane/ethyl acetate=9/1), thereby obtaining 14.2 g (yield 64%) of Compound C4 in the form of light-yellow crystals.

(3) The following reagents and solvent were charged into a flask with a capacity of 50 mL.
Compound C4: 1.95 g (8.51 mmol).
IrCl₃.3H₂O: 0.1 g (0.284 mmol).
Ethylene glycol: 20 mL.

A reflux pipe was then attached to the flask and the reaction liquid was rapidly heated in a microwave oven. The reflux was thereafter conducted for 7 min, while adjusting the microwave output. Once the reaction was completed, the reaction liquid, which was a red suspension, was cooled to about 120° C., and the crystals precipitated at this temperature were filtered. The crystals were successively washed with methanol, ethyl acetate, and isopropyl ether. The washed solids were the suspended in chloroform and washed by heating and refluxing. The washed solids were filtered and then vacuum dried to obtain 0.148 g of an Example Compound A1 (0.166 mmol, yield 58%) in the form of red crystals.

The molecular weight of the compound obtained was measured by MALDI-TOF-MAS (matrix-assisted laser desorption/ionization time-of-flight mass spectrometry) The molecular weight M⁺ of 876.2 was confirmed and the compound was identified as the Example Compound A-01.

The structure of this compound was verified by NMR measurements.

¹H-NMR (CDCl₃, 600 MHz) σ (ppm): 10.02 (s, 3H), 8.72 (d, 3H, J=8.5 Hz), 8.83 (d, 3H, J=8.8 Hz), 7.99 (d, 3H, J=8.8 Hz), 7.56 (t, 3H, J=7.7 Hz), 7.28 (d, 3H, J=7.7 Hz), 7.06 (t, 3H, J=7.5 Hz), 6.67 (d, 3H, J=7.7 Hz), 6.74 (t, 3H, J=7.5 Hz), 5.99 (d, 3H, J=7.5 Hz).

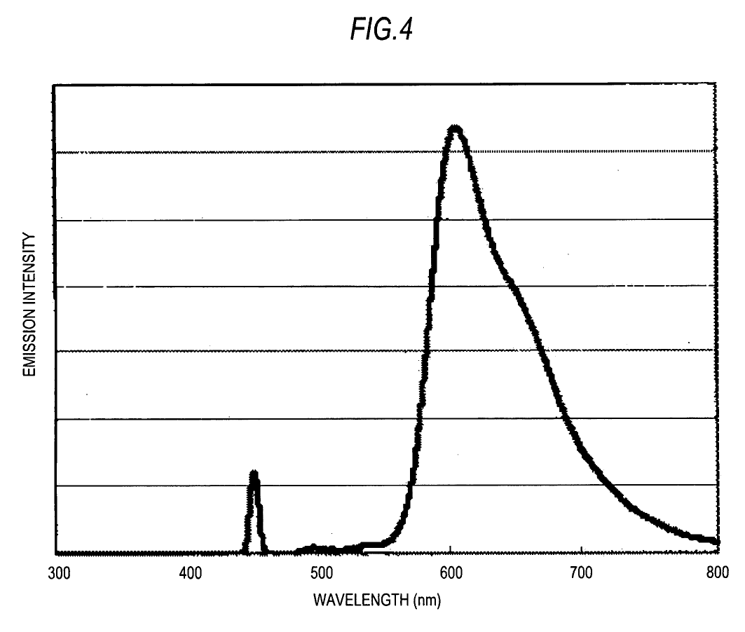

A 1×10⁻⁵ mol/l toluene solution of the Example Compound A1 was prepared and the light emission spectrum (PL spectrum) of the toluene solution was measured (excitation wavelength 510 nm). The light emission spectrum was measured by a method of photoluminescence measurements by using Hitachi F-4500. The PL spectrum shown in FIG. 4 was obtained in the measurements. The maximum peak wavelength of the PL spectrum was 607 nm.

### Example 2

### Synthesis of Example Compound B-04

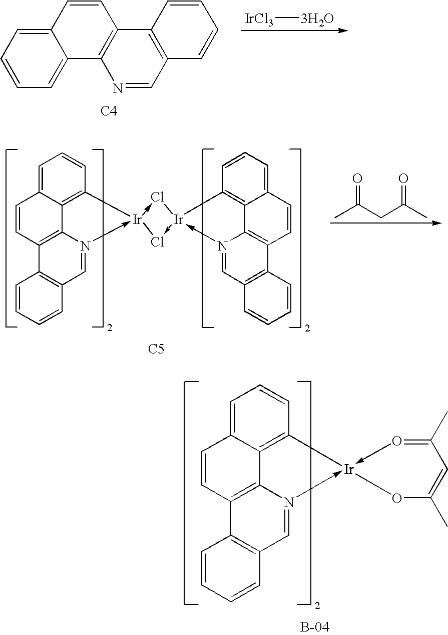

(1) The below described reagents and solvents were charged into a three-neck flask with a capacity of 200 mL.
Iridium (III) chloride trihydrate: 0.71 g (2 mmol)
Compound C-4: 1.83 g (8 mmol).
Ethoxyethanol: 90 ml.
Water: 30 ml.

The reaction liquid was then stirred for 30 min at room temperature under a nitrogen flow and then stirred for 10 h under refluxing. Upon completion of the reaction, the reaction solution was cooled to room temperature and the precipitated sediment was filtered. The sediment was then washed with water and then washed with ethanol. The washed sediment was vacuum dried at room temperature and 0.97 g (yield 71%) of compound C-5 in the form of a yellow-red powder was obtained.

(2) The following reagents and solvent were charged into a three-neck flask with a capacity of 200 mL.
Ethoxyethanol: 100 ml.
Compound C-5: 0.9 g (0.65 mmol).
Acetyl acetone: 0.2 g (2 mmol).
Sodium carbonate: 0.85 g (8 mmol).

The reaction solution was then stirred for 1 h at room temperature under a nitrogen flow, and then stirring was conducted for 7 h, while refluxing the reaction liquid. Upon completion of the reaction, the reaction solution was ice cooled. The precipitated sediment was filtered. The sediment was then washed with water and then washed with ethanol. The washed sediment was dissolved with chloroform and the insolubles were then filtered. The liquid obtained by such filtration was concentrated under reduced pressure and then recrystallized with a mixed chloroform-methanol solution. As a result, 0.39 g (yield 80%) of Example Compound B-04 was obtained in the form of a red powder.

The obtained Example Compound B-04 was dissolved in toluene and a 1×10⁻⁵ mol/l toluene solution was prepared. The PL spectrum of the toluene solution was measured by the same method as that of Example 1 (excitation wavelength 510 nm). As a result, the maximum peak wavelength of the PL spectrum was 610 nm.

### Example 3

An organic light-emitting element was produced in which an anode, a hole transport layer, a light emitting layer, an electron transport layer, and a cathode were provided in the order of description on a substrate.

The anode was formed by patterning ITO on the glass substrate. The anode film thickness in this case was 100 nm and the anode surface area was 3 mm².

The substrate with the ITO patterned thereon was transferred into a vacuum chamber under 10⁻⁵ Pa, and the below-described organic compound layer and electrode layer were continuously formed on the substrate by vacuum deposition using resistance heating. First, a Compound H-1 represented by the formula below was vapor deposited and a hole transport layer was formed. In this case, the thickness of the hole transport layer was 20 nm. A Compound H-2 represented by the formula below and serving as a host and the Example Compound A-1 as a guest were vapor co-deposited so as to obtain a weight concentration ratio thereof of 95:5, thereby forming the light-emitting layer. The thickness of the light-emitting layer in this case was 30 nm. A Compound H-3 represented by the formula below was then vapor deposited and the electron transport layer was formed. The thickness of the electron transport layer in this case was 30 nm. KF was then vapor deposited and a first metal electrode layer was formed. The thickness of the first metal electrode layer in this case was 1 nm. Al was then vapor deposited and a second metal electrode layer was formed. The thickness of the second metal electrode layer in this case was 100 nm. The first metal electrode layer and second metal electrode layer functioned as cathodes. An organic light-emitting element was thus obtained.

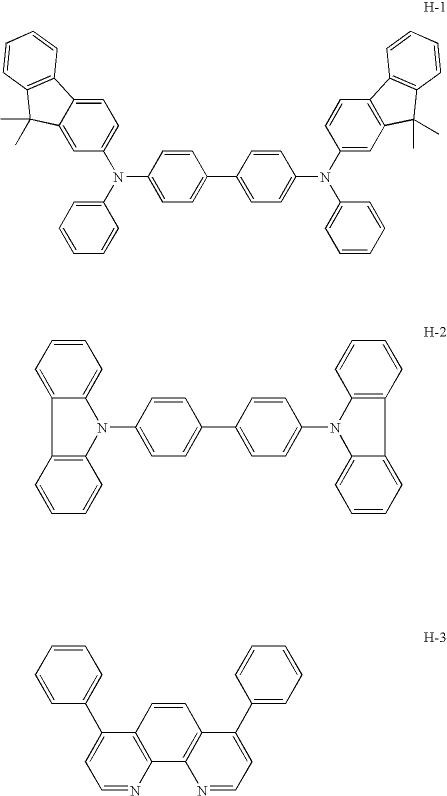

The current-voltage characteristic of the obtained organic light-emitting element was measured with a microampermeter 4140B manufactured by Hewlett-Packard Co. The emission luminance of the element was measured with BM7 manufactured by Topcon Corp. As a result, light emission derived from the Example Compound A-1 was detected from the element when a voltage of 6.0 V was applied.

The above described examples may be capable of providing a light-emitting organic element with good durability that has red emission of high efficiency and high-luminance.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2007-307693, filed Nov. 28, 2007, which is hereby incorporated by reference herein in its entirety.

What is claimed is:

1. An organometallic complex represented by Formula (1) below
MLₘL′ₙ (1)
wherein in Formula (1)
M stands for Ir, Rh, Pt, or Pd;
m is an integer of 1 to 3 and n is an integer of 0 to 2, where m+n=3;
MLₘ denotes a partial structure represented by Formula (2) below; and
ML′ₙ denotes a partial structure represented by at least one of Formulas (3) to (5) below 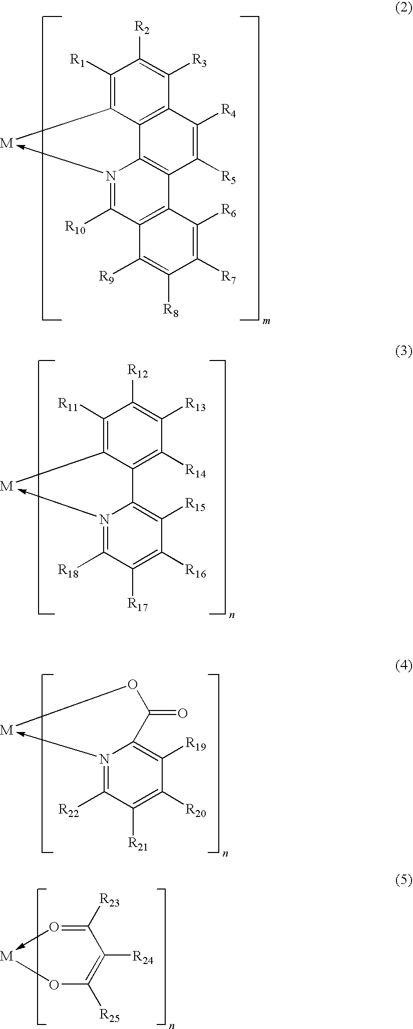
wherein in Formulas (2) to (5), R₁ to R₂₅, which may be the same or different, are each a hydrogen atom, a halogen atom, a substituted or unsubstituted alkyl group, a substituted or unsubstituted alkoxy group, a substituted or unsubstituted aryloxy group, an aralkyl group, a substituted amino group, an aryl group, or a heterocyclic group, and wherein any of substituents R₁ to R₂₅ that are adjacent to one another may optionally be bonded to each other, thereby forming a ring.

2. The organometallic complex according to claim 1, wherein the M is Ir.

3. An organic light-emitting element comprising
an anode and a cathode; and
a layer comprising an organic compound that is between the anode and the cathode, wherein
the layer comprising the organic compound includes the organometallic complex according to claim 1.

4. The organic light-emitting element according to claim 3, wherein
the layer comprising the organic compound is a light-emitting layer having the organometallic complex contained therein.

5. A display device comprising an organic light-emitting element according to claim 3 and means for supplying an electric signal to the organic light-emitting element.
